(19) United States
(12) Patent Application Publication
(10) Pub. No.: US 2026/0115532 A1
(43) Pub. Date: Apr. 30, 2026

(54) IoT-based respiratory measuring and training system and method through measurement of electromyography and respiratory pressure

(71) Applicant: INTHETECH Inc., Daegu (KR)

(72) Inventor: Seung Ho CHOUN, Daegu (KR)

(21) Appl. No.: 19/370,791

(22) Filed: Oct. 28, 2025

(30) Foreign Application Priority Data
Oct. 28, 2024 (KR) 10-2024-0148208

(51) Int. Cl. A63B 23/18; A61B 5/00; A61B 5/08; A61B 5/097; A61B 5/397; A63B 24/00

(52) U.S. Cl. CPC A63B 23/185; A61B 5/0022; A61B 5/0816; A61B 5/097; A61B 5/397; A61B 5/742; A63B 24/0075; A61B 2560/0431; A61B 2560/0443; A61B 2560/0456

(57) ABSTRACT

An IoT-based respiratory measuring and training system according to one embodiment of the present invention may include an IoT-based respiratory measuring device including a respiratory measuring module for measuring a user's respiration and an electromyography measuring module for measuring electromyography of a user's body part; and a user terminal for analyzing data on the measured respiration and electromyography to determine and output the user's respiratory pattern and breathing pattern.

### CROSS-REFERENCE TO RELATED APPLICATION

The present application claims priority under 35 U.S.C. § 119 (a) to Korean Patent application number 10-2024-0148208, filed on Oct. 28, 2024, in the Korean Intellectual Property Office, which is incorporated herein by reference in its entirety.

### BACKGROUND

The present invention relates to an IoT-based respiratory measuring and training system and method through the measurement of electromyography and respiratory pressure.

This research was supported by “Regional Innovation Strategy (RIS)” through the National Research Foundation of Korea (NRF) funded by the Ministry of Education (2022RIS-006).

COPD is a chronic obstructive pulmonary disease in which the airway narrows over a long period of time, causes chronic inflammation in the bronchi and lungs due to smoking, exposure to harmful gases in the workplace, indoor and outdoor air pollution, and lung infections, and causes difficulty in breathing as the airway narrows. COPD was ranked third among the causes of death worldwide in 2020 announced by the World Health Organization (WHO), and is expected to become the number one cause of death worldwide by 2050 due to air pollution and other factors.

In 2013, WHO designated fine dust as a carcinogen, and many researches have been presented showing a high correlation between acute lung damage and respiratory infections due to exposure to fine dust. Exposure to fine dust causes respiratory diseases such as acute pharyngitis or acute bronchitis, which cause symptoms such as sore throat, coughing, and phlegm. If these symptoms persist or recur, it can lead to a lung inflammatory response, and a persistent inflammatory response can lead to parenchymal destruction, which can cause emphysema and fibrosis of the small airway, which can lead to decreased lung function and chronic lung disease.

In addition, various after-effects of COVID-19, such as frequent coughing, fatigue, and sleep disorders, are appearing, and in particular, shortness of breath is considered one of the serious after-effects.

In the case of the aforementioned COPD and respiratory disease patients (including lung cancer and esophageal cancer), respiratory ability may be reduced during the treatment, so respiratory rehabilitation treatment including breathing methods such as abdominal breathing, sputum discharge induction, and light exercise prescription is required.

However, rehabilitation treatments require patients to visit the hospital in person, which can be inconvenient, and are typically available only during hospitalization. Furthermore, there is a lack of a structured system for ongoing disease management after discharge.

The matters described in the background of this invention are for understanding the background of the invention, and should not be construed as prior art known to a person of ordinary skill in the art to which this technology belongs.

### SUMMARY

The present invention is to provide an IoT-based respiratory measuring and training system and method through the measurement of electromyography and respiratory pressure, which enables the measurement of electromyography and respiratory pressure easily at home with a simple operation, thereby improving respiratory ability through home respiratory rehabilitation (respiratory management, exercise methods, and sputum discharge induction).

The present invention is to provide an IoT-based respiratory measuring and training system and method through the measurement of electromyography and respiratory pressure, which enables patients with decreased respiratory ability to continuously train regardless of location, and to improve their physical condition by measuring respiratory volume for oxygen supply, which is essential for muscle generation and nutrient supply.

Other objects of the present invention will be easily understood through the following description.

According to an aspect of the present invention, there is provided an IoT-based respiratory measuring and training system through the measurement of electromyography and respiratory pressure, including an IoT-based respiratory measuring device including a respiratory measuring module for measuring a user's respiration and an electromyography measuring module for measuring electromyography of a user's body; and a user terminal for analyzing data on the measured respiration and the electromyography to determine and output the user's respiratory pattern and breathing pattern.

The respiratory measuring module may include a module body graspable by the user; a mouth tip installed on an upper surface of the module body; a respiratory sensor disposed in a fluid passage formed through the module body, the respiratory sensor being configured to measure respiratory pressure according to the user's respiration through the mouth tip and to output respiratory measurement data; and a first module communication unit configured to transmit the respiratory measurement data to the user terminal.

The electromyography measuring module may include a lower body; a removable upper body on the lower body; an electromyography sensor including an electromyography measuring pad and a cable stored in the upper body; and a second communication module provided in the lower body and configured to receive electromyography measurement data measured by the electromyography measuring pad through the cable and to transmit the electromyography measurement data to the user terminal.

The IoT-based respiratory measuring device may further include a cradle configured to hold the respiratory measuring module and the electromyography measuring module in cradle grooves formed on an upper holding surface.

The user terminal may include a data processing unit configured to process the respiratory measurement data and the electromyography measurement data in a specified manner; a data analysis unit configured to analyze the processed respiratory measurement data and the electromyography measurement data to determine the user's respiratory pattern and breathing pattern; and an analysis result output unit configured to output a determination result through a display.

The user terminal may further include a respiratory guide unit configured to execute respiratory guidance content corresponding to the respiratory pattern and the breathing pattern to guide the user's respiration.

When the analysis result of the electromyography measurement data in the data analysis unit indicates a high dependency on respiratory muscles, the respiratory guide unit may execute a first respiratory guidance content to maintain a current respiratory pattern, and when the analysis result indicates a high dependency on accessory respiratory muscles, the respiratory guide unit may execute a second respiratory guidance content to guide abdominal breathing.

Meanwhile, according to another aspect of the present invention, there is provided an IoT-based respiratory measuring and training method performed in an IoT-based respiratory measuring and training system through the measurement of electromyography and respiratory pressure, including: acquiring a user's respiratory measurement data and electromyography measurement data through an IoT-based respiratory measuring device; processing the respiratory measurement data and the electromyography measurement data in a specified manner in a user terminal; analyzing the processed respiratory measurement data and the electromyography measurement data to determine the user's respiratory pattern and breathing pattern; and outputting the determination result through a display of the user terminal.

The method may further include executing respiratory guidance content corresponding to the respiratory pattern and the breathing pattern to guide the user's respiration.

When the analysis result of the electromyography measurement data in the data analysis unit indicates a high dependency on respiratory muscles, the respiratory guide unit may execute a first respiratory guidance content to maintain a current respiratory pattern, and when the analysis result indicates a high dependency on accessory respiratory muscles, the respiratory guide unit may execute a second respiratory guidance content to guide abdominal breathing.

Other aspects, features, and advantages in addition to those described above will become apparent from the following drawings, claims, and detailed description of the invention.

According to embodiments of the present invention, enabling the improvement of respiratory ability through home respiratory rehabilitation (respiratory management, exercise methods, sputum discharge induction) by easily measuring electromyography and respiratory pressure at home with a simple operation.

In addition, it is advantageous for patients with decreased respiratory ability because it enables them to continuously train regardless of location. It also enables physical improvement by measuring respiratory volume for oxygen supply, which is essential for muscle generation and nutrient supply.

The effects that can be obtained from the present invention are not limited to the effects mentioned above, and other unmentioned effects will be clearly understood by those with ordinary skill in the art to which the present invention belongs from the description below.

### BRIEF DESCRIPTION OF DRAWINGS

FIG. 1 is a block diagram showing the configuration of an IoT-based respiratory measuring and training system through the measurement of electromyography and respiratory pressure according to one embodiment of the present invention.

FIG. 2 is a perspective view of an IoT-based respiratory related measuring device according to an embodiment of the present invention.

FIG. 3 is a perspective view of a respiratory measuring module.

FIG. 4 is an exploded perspective view of a respiratory measuring module according to another embodiment.

FIG. 5 shows examples of a mouth tip.

FIG. 6 is a perspective view of an electromyography measuring module.

FIG. 7 is a top view, a front view, and a side view of a cradle.

FIG. 8 is an example view of an application screen linked to an IoT-based respiratory related measuring device.

FIG. 9 is a flowchart of an IoT-based respiratory measuring and training method through the measurement of electromyography and respiratory pressure according to one embodiment of the present invention.

FIG. 10 is a diagram illustrating the configuration of a system according to one embodiment of the present invention.

### DETAILED DESCRIPTION

The invention can be modified in various forms and specific embodiments will be described below and illustrated with accompanying drawings. However, the embodiments are not intended to limit the invention, but it should be understood that the invention includes all modifications, equivalents, and replacements belonging to the concept and the technical scope of the invention.

If it is mentioned that an element is “connected to” or “coupled to” another element, it should be understood that still another element may be interposed therebetween, as well as that the element may be connected or coupled directly to another element. On the contrary, if it is mentioned that an element is “connected directly to” or “coupled directly to” another element, it should be understood that still another element is not interposed therebetween.

Terms such as first, second, etc., may be used to refer to various elements, but, these elements should not be limited due to these terms. These terms will be used to distinguish one element from another element.

The terms used in the following description are intended to merely describe specific embodiments, but not intended to limit the invention. An expression of the singular number includes an expression of the plural number, so long as it is clearly read differently. The terms such as “include” and “have” are intended to indicate that features, numbers, steps, operations, elements, components, or combinations thereof used in the following description exist and it should thus be understood that the possibility of existence or addition of one or more other different features, numbers, steps, operations, elements, components, or combinations thereof is not excluded.

In addition, elements of an embodiment described below with reference to the accompanying drawings are not limited to the corresponding embodiment, may be included in another embodiment without departing from the technical spirit of the invention. Although particular description is not made, plural embodiments may be embodied as one embodiment.

In describing the invention with reference to the accompanying drawings, like elements are referenced by like reference numerals or signs regardless of the drawing numbers and description thereof is not repeated. If it is determined that detailed description of known techniques involved in the invention makes the gist of the invention obscure, the detailed description thereof will not be made.

In addition, the terms such as “ . . . part”, “ . . . unit”, “ . . . module”, “ . . . device”, etc. described in the specification mean a unit that processes at least one function or operation, and this can be implemented as a combination of hardware, software, or hardware and software.

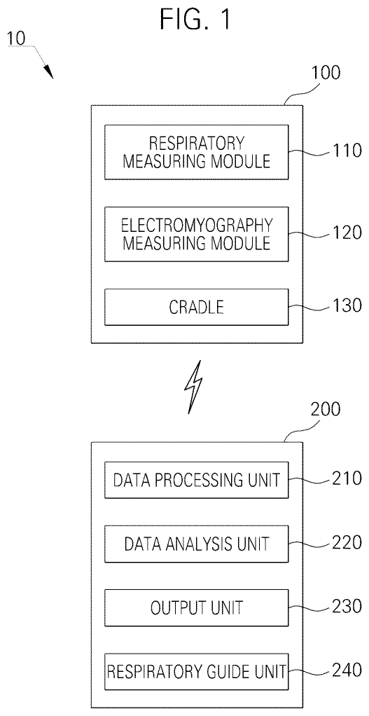

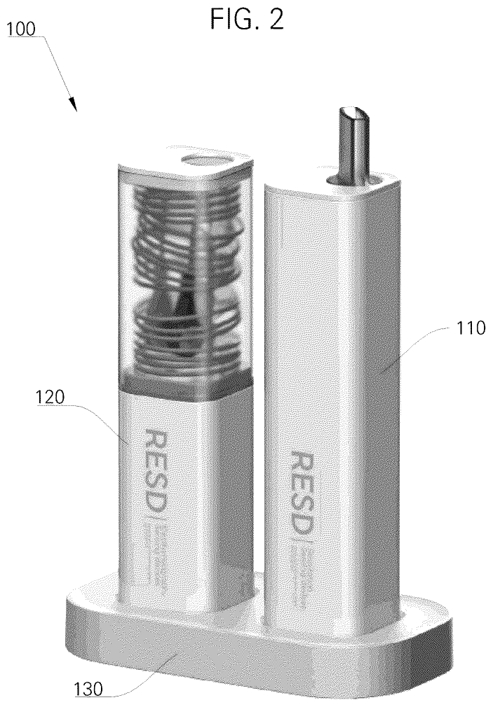

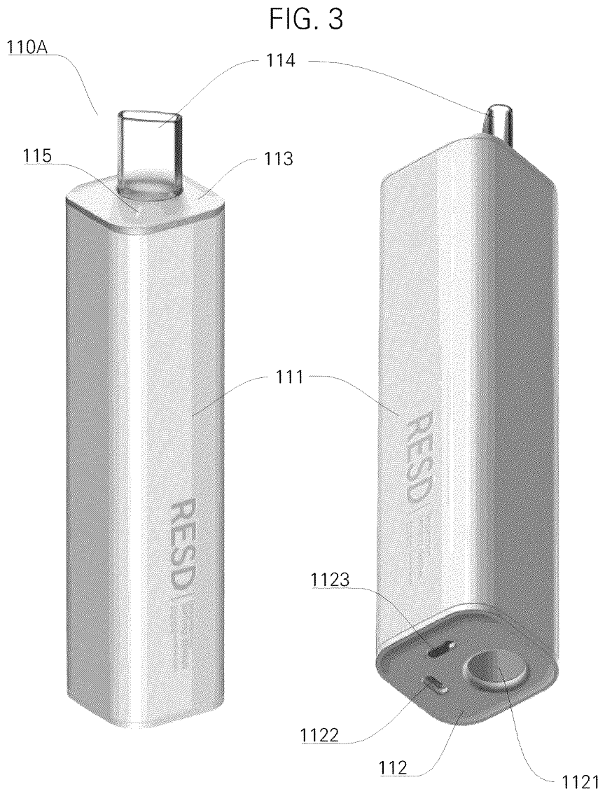

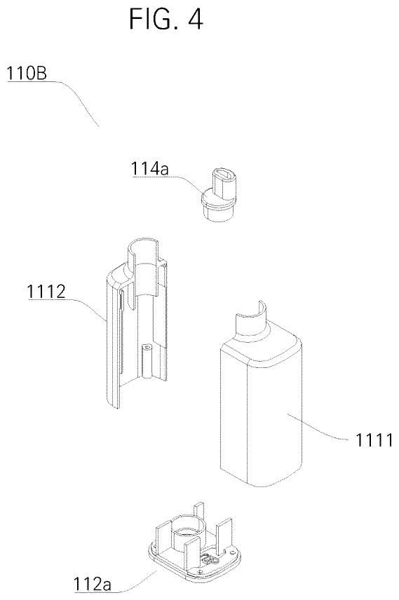

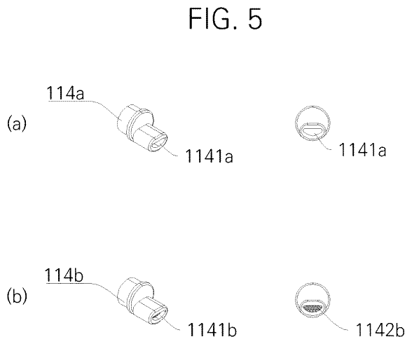

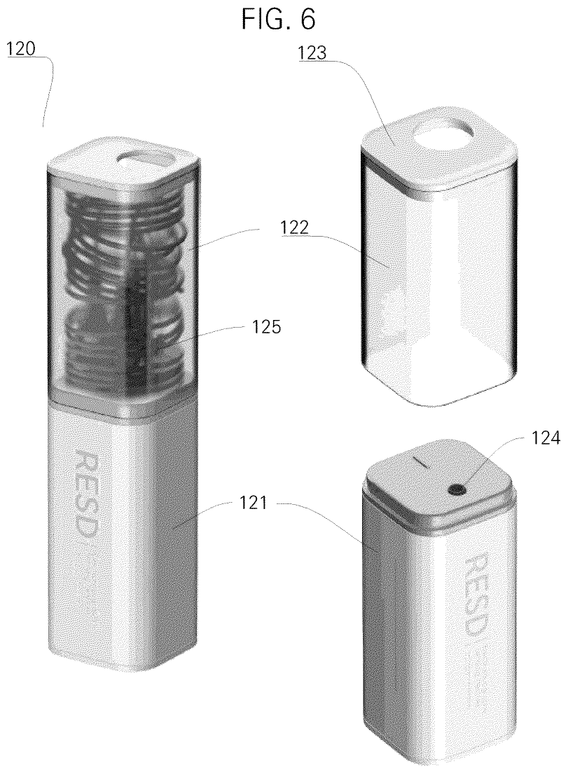

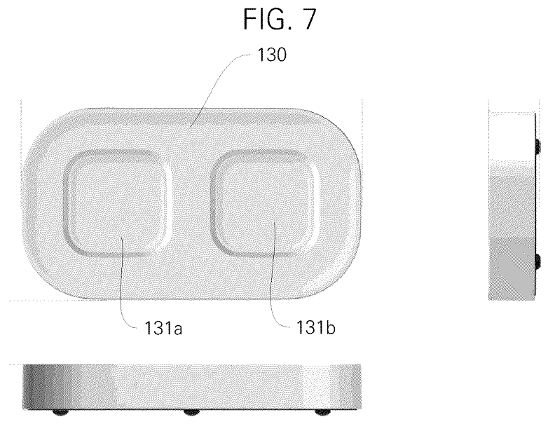

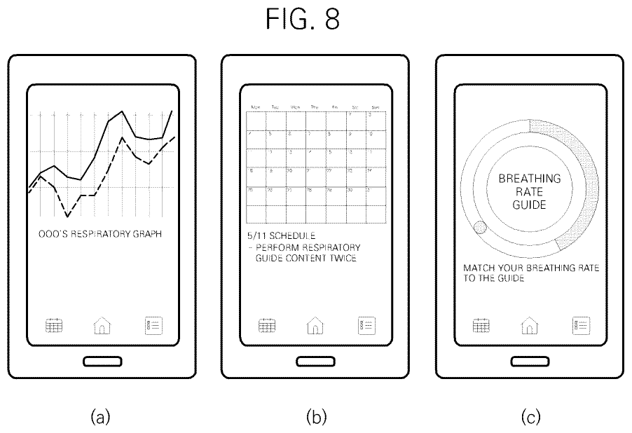

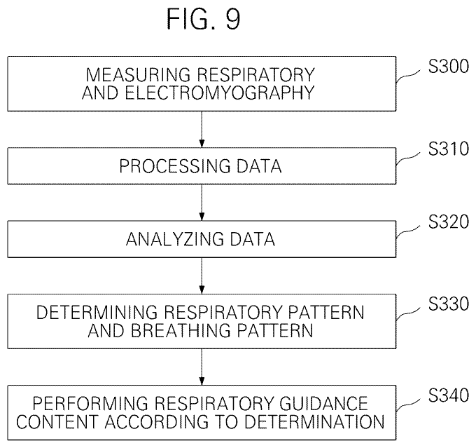

FIG. 1 is a block diagram showing the configuration of an IoT-based respiratory measuring and training system through the measurement of electromyography and respiratory pressure according to an embodiment of the present invention, FIG. 2 is a perspective view of an IoT-based respiratory related measuring device according to an embodiment of the present invention, FIG. 3 is a perspective view of a respiratory measuring module, FIG. 4 is an exploded perspective view of a respiratory measuring module according to another embodiment, FIG. 5 shows examples of a mouth tip, FIG. 6 is a perspective view of an electromyography measuring module, FIG. 7 is a top view, a front view, and a side view of a cradle, FIG. 8 is an example view of an application screen linked to an IoT-based respiratory related measuring device, and FIG. 9 is a flowchart of an IoT-based respiratory measuring and training method through the measurement of electromyography and respiration.

In FIGS. 1 to 8, an IoT-based respiratory measuring and training system 10, an IoT-based respiratory related measuring device 100, a respiratory measuring module 110A, 110B (hereinafter also collectively referred to as ‘110’), an electromyography measuring module 120, a cradle 130, a user terminal 200, a data processing unit 210, a data analysis unit 220, an analysis result output unit 230, a respiratory guide unit 240, a respiratory measuring module body 111, a lower plate 112, an upper plate 113, a mouth tip 114a, 114b (hereinafter also collectively referred to as ‘114’), an LED 115, a module intake/exhaust port 1121, a USB port 1122, a switch 1123, a first body 1111, a second body 1112, a tip intake/exhaust port 1141a, 1141b, a barrier 1142b, an electromyography measuring lower body 121, an electromyography measuring upper body 122, an upper plate 123, a cable connection terminal 124, and module cradle grooves 131a, 131b are shown.

In respiratory rehabilitation, exercise therapy is the most important element, and improved muscle function after exercise therapy can improve exercise ability even without improvement in lung function. It is an essential element of a rehabilitation program to improve exercise ability by restoring the physical strength of deteriorated skeletal muscles through a rehabilitation program.

The IoT-based respiratory measuring and training system 10 according to one embodiment of the present invention has a simple structure for home use, and is able to help a user (a respiratory patient, for example, COPD patient, a COVID-19 patient, etc.) who has difficulty breathing and shortness of breath during exercise, and needs respiratory measurement, management, and improvement through respiratory rehabilitation, thereby improving respiratory ability.

Referring to FIG. 1, the IoT-based respiratory measuring and training system 10 according to one embodiment may include an IoT-based respiratory related measuring device 100 and a user terminal 200.

The IoT-based respiratory related measuring device 100 is a hardware device configured to measure a user's respiratory volume (respiratory pressure) and/or electromyography.

The user terminal 200 is a terminal connected to the IoT-based respiratory related measuring device 100 through a network. The terminal is a computing device equipped with an linked application for IoT-based respiratory measurement and training, which is either pre-installed or can be downloaded and installed, and an operating system that can run the integrated application, and can be, for example, a smartphone, a tablet PC, a laptop, a desktop PC, etc.

The terminal may include an input unit (e.g., a touch screen, a keypad, a keyboard, a mouse, etc.) for receiving a user's input, an output unit (e.g., a display, a touch screen, etc.) for displaying various information, and a communication unit (e.g., a mobile communication module (3G, 4G, 5G, etc.), a short-range communication module (WiFi, Bluetooth, etc.), etc.) for communication with the IoT-based respiratory related measuring device 100.

The linked application can be installed on the user terminal 200 to process measurement data (respiratory volume, electromyography, etc.) for the user in a specified manner and display it through the output unit. In addition, it can guide the user to perform respiratory training according to a pre-set training program for the user in a manner perceivable by the user such as screen video output or audio output.

The IoT-based respiratory related measuring device 100 includes a respiratory measuring module 110 and an electromyography measuring module 120. In addition, the IoT-based respiratory related measuring device 100 may further include a cradle 130 for holding the respiratory measuring module 110 and the electromyography measuring module 120.

Referring to FIG. 3, the respiratory measuring module 110A includes a hollow, vertically elongated, respiratory measuring module body 111 as a basic frame. The respiratory measuring module body 111 can function as a handle for the user to hold during respiratory measurement or respiratory training.

An upper plate 113 and a lower plate 112 may be disposed at the top and bottom of the respiratory measuring module body 111, respectively. According to the embodiment, at least one of the upper plate 113 and the lower plate 112 may be integrally formed with the respiratory measuring module body 111.

A through-hole is formed in the upper plate 113, and a mouth tip 114 may be disposed in the through-hole. The mouth tip 114 is designed to be placed in the user's mouth when measuring respiration or performing respiratory training, and may be provided to be replaceable for hygiene.

A module intake/exhaust port 1121 may be formed at a vertical position corresponding to the mouth tip 114 of the upper plate 113. That is, a fluid passage, through which air (in particular, user's inhalation and/or exhalation) flows from the mouth tip 114 to the module intake/exhaust port 1121 is formed.

A sensor (e.g., a respiratory sensor) for measuring user respiration may be disposed in the fluid passage. The operation of the respiratory sensor can be performed as follows. Air enters the fluid passage. There is a mesh screen disposed in the fluid passage, and a pressure difference occurs between the top and bottom of the screen. The pressure difference between the top and bottom of the screen can be transmitted to a pressure transducer and measured. The greater the amount of air passing through the screen, the greater the pressure difference, so the air flow rate can be measured. The volume of air can be calculated by integrating the air flow rate as a function of time.

The sensor data can be transmitted to a module communication unit (not shown) provided in the respiratory measuring module body 111.

The module communication unit can transmit the sensor data (respiratory volume measurement data) transmitted from the sensor to the user terminal 200. The module communication unit is a wireless communication device capable of mobile communication or short-range communication, and can communicate with the communication unit of the user terminal 200.

An LED 115 may be disposed on the upper plate 113. The LED 115 can function as a status display unit that displays the status of the module. For example, a red light may indicate a charging state, a green light may indicate a fully charged state, a blue blinking light may indicate a communication pairing state, a blue solid light may indicate a pairing complete (normal operation) state, and a red blinking light may indicate a malfunction state.

In addition, a battery (not shown) may be mounted in the respiratory measuring module body 111. The battery can supply power to the sensor, the module communication unit, the LED 115, etc., ensure proper operation of each element.

The battery can be wirelessly charged by a cradle 130 with a wireless charging function. Alternatively, the battery may be wired-charged by being connected to a power supply device with a USB cable through a USB port 1122 provided in the lower plate 112.

In addition, a switch 1123 is disposed on the lower plate 112 to turn on/off the power of the respiratory measuring module 110. For example, the switch 1123 can be implemented as a slide type.

Referring to FIG. 4, another embodiment of the respiratory measuring module 110B is shown.

The body of the respiratory measuring module 110A may be separated into a first body 1111 and a second body 1112, and may be assembled on the lower plate 112a. An upper plate is not separately provided, and upper portions of the first body 1111 and the second body 1112 may form the upper plate.

And a mouth tip 114a can be inserted and coupled into an insertion groove made in the upper portions of the first body 1111 and the second body 1112.

Referring to (a) of FIG. 5, the first mouth tip 114a may have a form in which the first tip intake/exhaust port 1141a is fully open. This allows the user's respiration (inhale and/or exhale) to flow into the fluid passage as it is without any hindrance.

Referring to (b) of FIG. 5, a barrier 1142b may be disposed in the second tip intake/exhaust port 1141b of the second mouth tip 114b. The barrier 1142b allows most of the user's respiration to pass through, but prevents foreign substances (e.g., saliva, phlegm, etc.) discharged during the respiration process from flowing into the fluid passage, thereby enabling more accurate sensor data acquisition.

Referring to FIG. 6, the electromyography measuring module 120 may include an electromyography measuring module lower body 121 and an electromyography measuring module upper body 122.

The upper body 122 may be fitted and coupled onto the lower body 121 so as to be assembled into a single body and held in the cradle 130. When the lower body 121 and the upper body 122 are assembled, the assembly may have substantially the same height as the respiratory measuring module body 111, thereby providing balance when being held on the cradle 130.

A module communication unit for transmitting electromyography measurement data, a battery for supplying power, etc. may be in the lower body 121. The lower plate of the lower body 121 may be configured to include a USB port, a switch, etc., similar to the lower plate of the aforementioned respiratory measuring module body.

A cable connection terminal 124 is disposed on the upper surface of the lower body 121. The cable connection terminal 124 is a part to which a cable of an electromyography sensor 125 to be described later is connected, and data measured by the electromyography sensor 125 can be transmitted.

The upper body 122 has a hollow, elongated body. A device for electromyography measurement (electromyography sensor 125) may be disposed in the upper body 122. The electromyography sensor 125 includes an electromyography measuring pad attached to the user's body and a cable for providing an electrical signal.

In the case of the electromyography sensor 125, due to the presence of a cable of a predetermined length, it is not easy to store when not in use, and it can be frequently misplaced. In this embodiment, the upper body 122 functions as a storage case when not in use, so that the electromyography measuring pad and the cable can be stored in the internal space, thereby enabling easy storage and preventing loss. In addition, by having an appearance corresponding to the respiratory measuring module 110, it can provide an aesthetic appearance during storage.

The upper body 122 may be made of a transparent or translucent material. Therefore, by allowing the contents of the internal space to be checked, it allows the user to visually check whether the electromyography sensor 125 is stored normally or has been lost from the outside of the upper body 122.

The electromyography measuring pad of the electromyography sensor 125 is attached to the muscle area related to respiration. The muscle area related to respiration includes accessory respiratory muscles (sternocleidomastoid muscle). When the electromyography measuring pad is attached to the accessory respiratory muscles, the electromyography sensor 125 can measure and provide electromyography as related data in conjunction with respiration measured by the user using the respiratory measuring module 110.

The data measured by the electromyography sensor 125 can be analyzed by the user terminal 200 to check the dependency on accessory respiratory muscles and the breathing pattern during the user's respiration process. And based on the check result, a respiratory guide can be provided as a personalized respiratory rehabilitation content to assist the user in adopting proper breathing techniques.

Referring to FIG. 7, the cradle 130 is a device with a predetermined area of a holding surface. Cradle grooves 131a, 131b for holding the respiratory measuring module 110 and the electromyography measuring module 120 may be engraved on the holding surface. Therefore, the respiratory measuring module 110 and the electromyography measuring module 120 can be stably held on the cradle 130.

The cradle 130 may include a wireless charging module (not shown) inside. Therefore, when the respiratory measuring module 110 and/or the electromyography measuring module 120 are held in the cradle grooves 131a, 131b, the wireless charging module can operate to wirelessly charge the batteries of the respiratory measuring module 110 and/or the electromyography measuring module 120.

The user terminal 200 may include a data processing unit 210, a data analysis unit 220, and an analysis result output unit 230. It may further include a respiratory guide unit 240 as needed. The data processing unit 210, the data analysis unit 220, the analysis result output unit 230, and the respiratory guide unit 240 of the user terminal 200 can be implemented as software, and two or more units may be integrated or one unit may be divided into two or more units and implemented.

When the user breathes using the respiratory measuring module 110, the user's respiration can be measured. In addition, when the electromyography sensor 125 is attached to the user's body (accessory respiratory muscle area) using the electromyography measuring module 120 during the respiration process, electromyography can also be measured (step S300). The measured data can be transmitted to the user terminal 200 through the module communication unit.

The data processing unit 210 is configured to receive the data, i.e., the respiratory measurement data and the electromyography measurement data, transmitted from the module communication unit of the respiratory measuring module 110 and/or the module communication unit of the electromyography measuring module 120, and processes the data in a specified manner (step S310). The data processing method can be determined according to the characteristics of the respiratory sensor and the electromyography sensor, which are well-known to one with ordinary skill in the art to which the present invention belongs, so a detailed description thereof will be omitted.

The data analysis unit 220 is configured to analyze information about the user's respiration from the processed respiratory measurement data and electromyography measurement data (step S320).

The user respiration information may be data over time. In addition, the user's respiration and electromyography measured at the same time can be matched and analyzed.

The analysis result output unit 230 may be configured to cause the analysis result from the data analysis unit 220 to be displayed on the terminal screen. Referring to (a) of FIG. 8, the user's respiration graph is displayed chronologically. When displaying the user's respiration graph (dotted line), a normal respiration graph (solid line) can be displayed together to allow a visual and easy check of whether the respiratory pattern is normal.

Based on the respiratory pattern according to the analysis result of the respiratory measurement data, the respiratory guide unit 240 may be configured to distinguish between a case where the dependency on respiratory muscles is high and a case where the dependency on accessory respiratory muscles is high when the user breathes according to the analysis result of the electromyography measurement data (step S330).

In the case of a high dependency on respiratory muscles, it can be determined that a regular and stable respiratory pattern is being maintained. In this case, the respiratory pattern is efficient and it is highly likely that no correction is needed.

In the case of a high dependency on accessory respiratory muscles, it can be determined as an irregular respiratory pattern. In this case, it is likely to be shallow chest breathing, which often appears in a tense state.

The respiratory guide unit 240 may be configured to execute respiratory guidance content corresponding to the respiratory pattern and breathing pattern determined for the user, and cause the user to perform respiration in a normal manner (step S340).

In the case of a high dependency on respiratory muscles, since normal respiration is already being performed, the respiratory guide unit 240 may be configured to guide the user to maintain the current respiratory pattern or execute first respiratory guidance content that recommends deeper and slower abdominal breathing to induce more efficient respiration.

In the case of a high dependency on accessory respiratory muscles, in order to correct inefficient breathing patterns of excessively using the accessory respiratory muscles, the respiratory guide unit 240 may be configured to execute second respiratory guidance content that guides diaphragmatic breathing.

When the second respiratory guidance content is executed, the respiratory measuring module 110 can be operated as follows.
Guiding deep breathing: giving the user a notification to breathe deeply and regularly or suggesting a breathing exercise.
Adjusting respiratory pattern: providing a guide or real-time feedback to the user to perform abdominal breathing. For example, guiding the user to slowly inhale through the nose and inflate the belly.

An example of respiratory guidance content is shown in (c) of FIG. 8.

An exemplary screen that shows the history of performing the respiratory guidance content in a calendar format is shown in (b) of FIG. 8.

According to the IoT-based respiratory volume measuring system 10 according to this embodiment, it is possible to improve the user's physical condition by measuring respiratory volume for oxygen supply, which is essential for muscle generation and nutrient supply. It enables patients with decreased respiratory ability to continuously train regardless of location. In the case of COPD patients, it can be used for the purpose of measuring respiratory volume and removing secretions in the airway.

In addition, ordinary users can carry the system 10 during daily physical activities and use the measured respiratory volume as a reference indicator. And the system 10 has versatility that can simultaneously satisfy two purposes: improving the respiration of athletes and measuring and rehabilitating respiration for patients with respiratory system diseases.

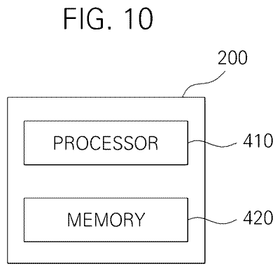

FIG. 10 is a diagram illustrating the configuration of a system according to an embodiment of the present invention.

Referring to FIG. 10, the user terminal 200 of the IoT-based respiratory measuring and training system 10 includes a processor (410) and a memory (420). The memory (420) stores one or more instructions executable by the processor (410). The processor (410) executes one or more instructions stored in the memory (420). The processor (410) can execute one or more operations described above in connection with FIG. 8 by executing the instructions. In addition, the configuration of the present invention described above with reference to FIG. 1 may be a configuration implemented by instructions executed by the processor (410).

The embodiments described above can be implemented as hardware components, software components, and/or a combination of hardware and software components. For example, the devices, methods, and elements described in the embodiments can be implemented using one or more general-purpose computers or special-purpose computers, such as a processor, a controller, a Central Processing Unit (CPU), a Graphics Processing Unit (GPU), an arithmetic logic unit (ALU), a digital signal processor, a microcomputer, a field programmable gate array (FPGA), a programmable logic unit (PLU), a microprocessor, an Application Specific Integrated Circuit (ASICS), or any other device that can execute and respond to instructions.

The aforementioned IoT-based respiratory measuring and training method can also be implemented in the form of a recording medium including computer-executable instructions, such as an application or program module executed by a computer. A computer-readable medium may be any available medium that can be accessed by a computer, and includes both volatile and non-volatile media, and both removable and non-removable media. In addition, the computer-readable medium may include a computer storage medium. A computer storage medium includes both volatile and non-volatile, removable and non-removable media implemented by any method or technology for storing information, such as computer-readable instructions, data structures, program modules, or other data.

The aforementioned IoT-based respiratory measuring and training method can be executed by an application (which may include a program included in a platform or operating system mounted on the terminal by default) installed on the terminal by default, and can also be executed by an application (i.e., a program) installed directly on the master terminal by the user through an application store server or an application providing server such as a web server related to the application or the service. In this sense, the aforementioned IoT-based respiratory measuring and training method is implemented as an application (i.e., a program) installed on the terminal by default or installed directly by the user, and can be recorded on a computer-readable recording medium such as a terminal.

Although the embodiments of the present invention have been described above with reference to the drawings, those skilled in the art will understand that various modifications and changes can be made to the present invention without departing from the spirit and scope of the present invention as described in the following claims.

What is claimed is:

1. An IoT-based respiratory measuring and training system through the measurement of electromyography and respiratory pressure, comprising:
an IoT-based respiratory measuring device including a respiratory measuring module for measuring a user's respiration and an electromyography measuring module for measuring electromyography of a user's body; and
a user terminal for analyzing data on the measured respiration and the electromyography to determine and output the user's respiratory pattern and breathing pattern.

2. The IoT-based respiratory measuring and training system of claim 1, wherein the respiratory measuring module comprises:
a module body graspable by the user;
a mouth tip installed on an upper surface of the module body;
a respiratory sensor disposed in a fluid passage formed through the module body, the respiratory sensor being configured to measure respiratory pressure according to the user's respiration through the mouth tip and to output respiratory measurement data; and
a first module communication unit configured to transmit the respiratory measurement data to the user terminal.

3. The IoT-based respiratory measuring and training system of claim 2, wherein the electromyography measuring module comprises:
a lower body;
a removable upper body on the lower body;
an electromyography sensor including an electromyography measuring pad and a cable stored in the upper body; and
a second communication module provided in the lower body and configured to receive electromyography measurement data measured by the electromyography measuring pad through the cable and to transmit the electromyography measurement data to the user terminal.

4. The IoT-based respiratory measuring and training system of claim 3 further comprising a cradle configured to hold the respiratory measuring module and the electromyography measuring module in cradle grooves formed on an upper holding surface.

5. The IoT-based respiratory measuring and training system of claim 3, wherein the user terminal comprises:
a data processing unit configured to process the respiratory measurement data and the electromyography measurement data in a specified manner;
a data analysis unit configured to analyze the processed respiratory measurement data and the processed electromyography measurement data to determine the user's respiratory pattern and breathing pattern; and
an analysis result output unit configured to output a determination result through a display.

6. The IoT-based respiratory measuring and training system of claim 5, wherein the user terminal further comprises a respiratory guide unit configured to execute respiratory guidance content corresponding to the respiratory pattern and the breathing pattern to guide the user's respiration.

7. The IoT-based respiratory measuring and training system of claim 6, wherein when the analysis result of the electromyography measurement data in the data analysis unit indicates a high dependency on respiratory muscles, the respiratory guide unit executes a first respiratory guidance content to maintain a current respiratory pattern, and when the analysis result indicates a high dependency on accessory respiratory muscles, the respiratory guide unit executes a second respiratory guidance content to guide abdominal breathing.

8. An IoT-based respiratory measuring and training method performed in an IoT-based respiratory measuring and training system through the measurement of electromyography and respiratory pressure, comprising:
acquiring a user's respiratory measurement data and electromyography measurement data through an IoT-based respiratory measuring device;
processing the respiratory measurement data and the electromyography measurement data in a specified manner in a user terminal;
analyzing the processed respiratory measurement data and the electromyography measurement data to determine the user's respiratory pattern and breathing pattern; and
outputting the determination result through a display of the user terminal.

9. The IoT-based respiratory measuring and training method of claim 8, further comprising executing respiratory guidance content corresponding to the respiratory pattern and the breathing pattern to guide the user's respiration.

10. The IoT-based respiratory measuring and training method of claim 9, wherein when the analysis result of the electromyography measurement data indicates a high dependency on respiratory muscles, the respiratory guide unit executes a first respiratory guidance content to maintain the current respiratory pattern, and when the analysis result indicates a high dependency on accessory respiratory muscles, the respiratory guide unit executes a second respiratory guidance content to guide abdominal breathing.
